(19) United States
(12) Patent Application Publication
(10) Pub. No.: US 2026/0144527 A1
(43) Pub. Date: May 28, 2026

(54) ULTRASONIC PROBE AND CONTROL METHOD THEREOF

(71) Applicant: SAMSUNG MEDISON CO., LTD., Hongcheon (KR)

(72) Inventors: Hwanjo Lee, Hongcheon (KR); Hwanseung Yu, Hongcheon (KR); Namwoong Kim, Hongcheon (KR); Jungmin Kim, Hongcheon (KR)

(21) Appl. No.: 19/170,090

(22) Filed: Apr. 4, 2025

(30) Foreign Application Priority Data
Nov. 28, 2024 (KR) 10-2024-0173909

(51) Int. Cl. A61B 8/00; G01S 7/52

(52) U.S. Cl. CPC A61B 8/54; A61B 8/4477; A61B 8/469; G01S 7/52057

(57) ABSTRACT

Disclosed is an ultrasonic probe including a first transducer configured to transmit a first transmission signal and receive a reflection signal, a second transducer configured to transmit a second transmission signal and receive a reflection signal, a first pulser configured to supply an electrical signal to the first transducer, a second pulser configured to supply an electrical signal to the second transducer, a voltage generator configured to supply a voltage to the first pulser and the second pulser, and a controller configured to supply a voltage generated in the voltage generator to the second pulser to discharge a voltage supplied to the first pulser when an image mode is changed in a state in which the first transducer is activated.

### CROSS-REFERENCE TO RELATED APPLICATION(S)

This application is based on and claims priority under 35 U.S.C. § 119 to Korean Patent Application No. 10-2024-0173909, filed on Nov. 28, 2024, in the Korean Intellectual Property Office, the disclosure of which is incorporated by reference herein in its entirety.

### BACKGROUND

### 1. Field

The disclosure relates to an ultrasonic probe emitting ultrasonic waves to an object and receives echo ultrasonic waves reflected from the object, and a control method thereof.

### 2. Description of the Related Art

Recently, in a medical field, various medical imaging apparatuses have been widely used to image and obtain information about biological tissues of a human body for the purpose of early diagnosis of various diseases or surgery. Representative examples of such medical imaging apparatuses may include ultrasonic imaging apparatuses, computed tomography (CT) apparatuses, and magnetic resonance imaging (MRI) apparatuses.

An ultrasonic imaging apparatus is a device that emits an ultrasonic signal generated from a transducer of a probe to an object, and non-invasively obtains at least one image of a region inside the object (e.g., soft tissue or blood flow) by receiving information from the signal reflected from the object. In particular, an ultrasonic imaging apparatus is used for medical purposes such as observing the inside of an object, detecting foreign substances, and measuring injury. Such an ultrasonic imaging apparatus is widely used together with other imaging diagnostic apparatuses because the ultrasonic imaging apparatus has higher stability than an imaging apparatus using an X-ray, may display images in real time, and is safe because there is no radiation exposure.

As described above, an image of a target region may be obtained through a probe connected to a main body by a wire, but in order to obtain an image of the target region regardless of time and place, an ultrasonic probe being operated wirelessly is required.

In the case of a probe being operated wirelessly, because most of operations performed in a main body need to be performed in the probe and thus there are many spatial constraints in design, research and development to overcome these constraints has been actively conducted recently.

In addition, in the case of wireless ultrasonic imaging apparatuses, a structure of dual heads configured on opposite sides of different shapes is being adopted in many cases in consideration of user convenience.

### SUMMARY

It is an aspect of the disclosure to provide an ultrasonic probe and a control method thereof capable of rapidly changing an image mode by discharging a voltage generator without a separate discharge module.

Technical tasks to be achieved in this document are not limited to the technical task above, and other technical tasks not mentioned will be clearly understood by those skilled in the art from the description below.

An aspect of the disclosure provides an ultrasonic probe including a first transducer configured to transmit a first transmission signal and receive a reflection signal, a second transducer configured to transmit a second transmission signal and receive a reflection signal, a first pulser configured to supply an electrical signal to the first transducer, a second pulser configured to supply an electrical signal to the second transducer, a voltage generator configured to supply a voltage to the first pulser and the second pulser, and a controller configured to supply a voltage generated in the voltage generator to the second pulser to discharge a voltage supplied to the first pulser when an image mode is changed in a state in which the first transducer is activated.

An aspect of the disclosure provides a control method of an ultrasonic probe, which includes a first transducer configured to transmit a first transmission signal and receive a reflection signal, a second transducer configured to transmit a second transmission signal and receive a reflection signal, a first pulser configured to supply an electrical signal to the first transducer, a second pulser configured to supply an electrical signal to the second transducer, and a voltage generator configured to supply a voltage to the first pulser and the second pulser, including activating the first transducer, receiving a command for a change of an image mode, and supplying a voltage generated in the voltage generator to the second pulser to discharge a voltage supplied to the first pulser.

According to the disclosure, the voltage generator can be discharged without a separate discharge module so that the image mode can be rapidly changed.

In addition, because there is no discharge module, a volume and weight occupied by the ultrasonic probe can be reduced, and a voltage suitable for the changed image mode can be applied, so that the image quality can be secured.

### BRIEF DESCRIPTION OF THE DRAWINGS

These and/or other aspects of the disclosure will become apparent and more readily appreciated from the following description of the embodiments, taken in conjunction with the accompanying drawings of which:

FIGS. 1A and 1B are block diagrams illustrating components of an ultrasonic imaging system according to an embodiment;

FIGS. 2A, 2B, 2C, and 2D are views illustrating an ultrasonic imaging apparatus according to an embodiment;

FIG. 3 is a view illustrating an external appearance of an ultrasonic probe according to an embodiment;

FIG. 4 is a control block diagram of the ultrasonic probe according to an embodiment;

FIG. 5 is a diagram schematically illustrating a circuit structure of the ultrasonic probe according to an embodiment. ;

FIGS. 6 and 7 are flowcharts illustrating control methods of the ultrasonic probe according to an embodiment;

FIG. 8 is a diagram for explaining a movement of an electrical signal according to a change of an image mode according to an embodiment;

FIGS. 9A and 9B are diagrams for explaining a required voltage according to the change of the image mode according to an embodiment; and

FIGS. 10A and 10B are graphs for explaining a discharge time of a pulsar according to an embodiment.

### DETAILED DESCRIPTION

This disclosure will explain the principles and disclose embodiments of the disclosure to clarify the scope of the claims of the disclosure and enable those skilled in the art to which the embodiments of the disclosure belong to practice the embodiments. The embodiments of the disclosure may be implemented in various forms.

Throughout the specification, like reference numbers refer to like elements throughout this specification. This specification does not describe all components of the embodiments, and general contents in the technical field to which the disclosure belongs or overlapping contents between the embodiments will not be described. The “module” or “unit” used in the specification may be implemented as one or a combination of two or more of software, hardware, or firmware, and according to embodiments, a plurality of “module” or “unit” may be implemented as a single element, or a single “module” or “unit” may include a plurality of elements.

The singular form of a noun corresponding to an item may include a single item or a plurality of items, unless the relevant context clearly indicates otherwise.

In this disclosure, each of phrases such as “A or B,” “at least one of A and B,” “at least one of A or B,” “A, B or C,” “at least one of A, B and C,” and “at least one of A, B, or C” may include any one of the items listed together in the corresponding one of the phrases, or all possible combinations thereof.

The term “and/or” includes any combination of a plurality of related components or any one of a plurality of related components.

The terms such as “first,” “second,” “primary,” and “secondary” may simply be used to distinguish a given component from other corresponding components, and do not limit the corresponding components in any other respect (e.g., importance or order).

The terms “front surface,” “rear surface,” “upper surface,” “lower surface,” “side surface,” “left side,” “right side,” “upper portion,” “lower portion,” and the like used in the disclosure are defined with reference to the drawings, and the shape and position of each component are not limited by these terms.

The terms “comprises,” “has,” and the like are intended to indicate that there are features, numbers, steps, operations, components, parts, or combinations thereof described in the disclosure, and do not exclude the presence or addition of one or more other features, numbers, steps, operations, components, parts, or combinations thereof.

When any component is referred to as being “connected,” “coupled,” “supported,” or “in contact” with another component, this includes a case in which the components are indirectly connected, coupled, supported, or in contact with each other through a third component as well as directly connected, coupled, supported, or in contact with each other.

When any component is referred to as being located “on” or “over” another component, this includes not only a case in which any component is in contact with another component but also a case in which another component is present between the two components.

Hereinafter, an ultrasonic apparatus according to various embodiments will be described in detail with reference to the accompanying drawings. When described with reference to the attached drawings, similar reference numbers may be assigned to identical or corresponding components and redundant description thereof may be omitted.

In this disclosure, an image may include a medical image obtained by a medical imaging apparatus such as a magnetic resonance imaging (MRI) apparatus, a computed tomography (CT) apparatus, an ultrasonic imaging apparatus, or an X-ray imaging apparatus.

In this disclosure, an ‘object’, which is subject to photography, may include a person, animal, or part thereof. For example, the object may include a part of a human body (an organ, etc.) or a phantom.

In this disclosure, an ‘ultrasonic image’ refers to an image of an object that has been generated or processed based on an ultrasonic signal (echo signal) transmitted to and reflected from the object.

Hereinafter, embodiments will be described in detail with reference to the drawings.

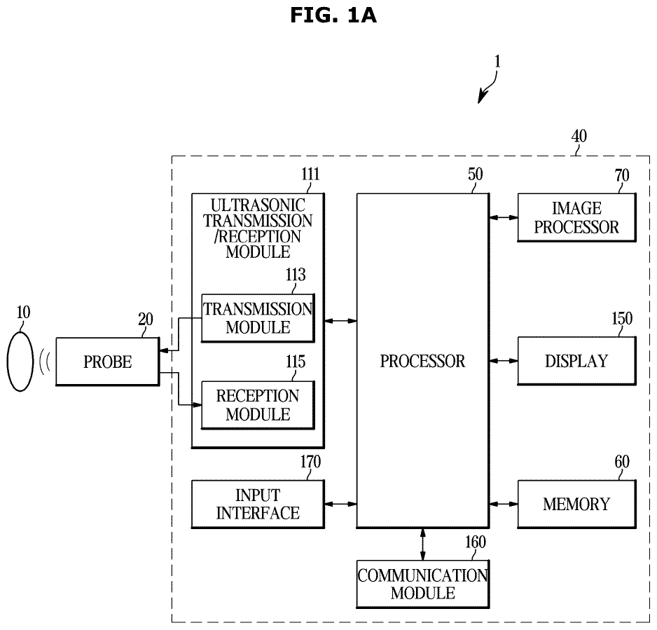

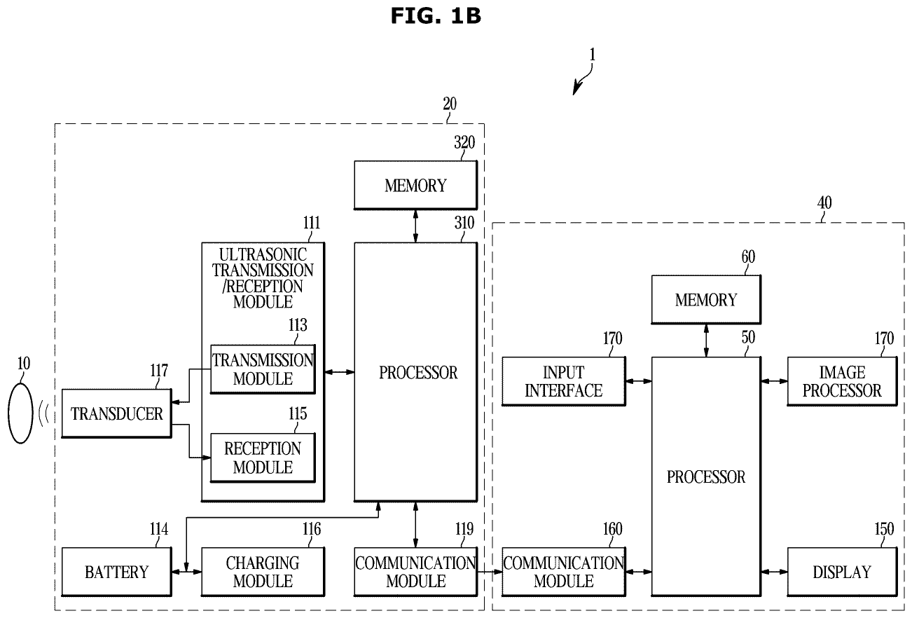

FIGS. 1A and 1B are block diagrams illustrating components of an ultrasonic imaging system according to an embodiment.

Referring to FIGS. 1A and 1B, an ultrasonic imaging system 1 may include a probe 20 and an ultrasonic imaging apparatus 40.

The ultrasonic imaging apparatus 40 may be implemented not only in a cart type but also in a portable type. A portable ultrasonic imaging apparatus may include, for example, a smart phone, a laptop computer, a personal digital assistant (PDA), a tablet PC, etc., which include a probe and an application, but is not limited thereto.

The probe 20 may include a wired probe connected to the ultrasonic imaging apparatus 40 by wire to communicate with the ultrasonic imaging apparatus 40 by wire, a wireless probe wirelessly connected to the ultrasonic imaging apparatus 40 to communicate wirelessly with the ultrasonic imaging apparatus 40, and/or a hybrid probe wire or wirelessly connected to the ultrasonic imaging apparatus 40 to communicate wire or wirelessly with the ultrasonic imaging apparatus 40.

According to various embodiments, as illustrated in FIG. 1A, the ultrasonic imaging apparatus 40 may include an ultrasonic transmission/reception module 111, or as illustrated in FIG. 1B, the probe 20 may include the ultrasonic transmission/reception module 111. According to various embodiments, both the ultrasonic imaging apparatus 40 and the probe 20 may also include the ultrasonic transmission/reception module 111.

According to various embodiments, the probe 20 may further include an image processor 70, a display 150, and/or an input interface 170.

Accordingly, descriptions of the ultrasonic transmission/reception module 111, the image processor 70, the display 150, and/or the input interface 170 included in the ultrasonic imaging apparatus 40 may also be applied to the ultrasonic transmission/reception module 111, the image processor 70, the display 150, and/or the input interface 170 included in the probe 20.

FIG. 1A illustrates a control block diagram of the ultrasonic imaging system 1 in a case in which the probe 20 is a wired probe or a hybrid probe.

The probe 20 may include a plurality of transducers. The plurality of transducers may transmit an ultrasonic signal to an object 10 in response to a transmission signal applied from a transmission module 113. The plurality of transducers may form a reception signal by receiving the ultrasonic signal (echo signal) reflected from the object 10. The probe 20 may be implemented as an integrated type with the ultrasonic imaging apparatus 40, or may be implemented as a separate type connected to the ultrasonic imaging apparatus 40 by wire. The ultrasonic imaging apparatus 40 may be connected to the one or more probes 20 depending on the implementation type.

In the case in which the probe 20 is a wired probe or a hybrid probe, the probe 20 may include a cable and a connector capable of being connected to a connector of the ultrasonic imaging apparatus 40.

The probe 20 according to an embodiment may be implemented as a two-dimensional probe. In a case in which the probe 20 is implemented as a two-dimensional probe, the plurality of transducers included in the probe 20 may be arranged in two dimensions to form a two-dimensional transducer array.

For example, the two-dimensional transducer array may have a form in which a plurality of sub-arrays including the plurality of transducers arranged in a first direction is arranged in a second direction different from the first direction.

In addition, in the case in which the probe 20 according to an embodiment is implemented as a two-dimensional probe, the ultrasonic transmission/reception module 111 may include an analog beamformer and a digital beamformer. Alternatively, the two-dimensional probe may include one or both of the analog beamformer and the digital beamformer depending on the implementation type.

A processor 50 controls the transmission module 113 to form a transmission signal to be applied to each of transducers 117 in consideration of positions and focused points of the plurality of transducers included in the probe 20.

the processor 50 may control a reception module 115 to generate ultrasonic data by converting reception signals received from the probe 20 into analog to digital and summing up the digitally converted reception signals in consideration of the positions and focused points of the plurality of transducers.

In the case in which the probe 20 is implemented as a two-dimensional probe, the processor 50 may calculate a time delay value for digital beamforming for each sub-array for each of the plurality of sub-arrays included in the two-dimensional transducer array. The processor 50 may also calculate a time delay value for analog beamforming for each of the transducers included in one of the plurality of sub-arrays. The processor 50 may control the analog beamformer and the digital beamformer to form a transmission signal to be applied to each of the plurality of transducers depending on the time delay values for analog beamforming and the time delay values for digital beamforming. The processor 50 may also control the analog beamformer to sum up the signals received from the plurality of transducers for each sub-array depending on the time delay values for analog beamforming. The processor 50 may also control the ultrasonic transmission/reception module 111 to convert the summed signal for each sub-array into analog to digital. The processor 50 may also control the digital beamformer to generate ultrasonic data by summing up the digitally converted signals depending on the time delay values for digital beamforming.

The image processor 70 generates an ultrasonic image using the generated ultrasonic data.

The display 150 may display the generated ultrasonic image and a variety of information processed in the ultrasonic imaging apparatus 40 and/or the probe 20. The probe 20 and/or the ultrasonic imaging apparatus 40 may include the one or more displays 150 depending on the implementation type. The display 150 may also include a touch panel or a touch screen.

The processor 50 may control the overall operation of the ultrasonic imaging apparatus 40 and signal flows between internal components of the ultrasonic imaging apparatus 40. The processor 50 may perform or control various operations or functions of the ultrasonic imaging apparatus 40 by executing programs or instructions stored in memory 60. The processor 50 may also control an operation of the ultrasonic imaging apparatus 40 by receiving a control signal from the input interface 170 or an external device.

The ultrasonic imaging apparatus 40 may include a communication module 160, and may be connected with the external device (e.g., the probe 20, a server, a medical device, a portable device (a smart phone, tablet PC, wearable device, etc.)) through the communication module 160.

The communication module 160 may include one or more components enabling communication with the external device, and may include, for example, at least one of a short-range communication module, a wired communication module, and a wireless communication module.

The communication module 160 may receive a control signal and data from the external device and transmit the received control signal to the processor 50 so that the processor 50 controls the ultrasonic imaging apparatus 40 in response to the received control signal.

Alternatively, the processor 50 may transmit a control signal to the external device through the communication module 160 to control the external device according to the control signal of the processor 50.

For example, the external device may process the data of the external device according to the control signal of the processor 50 received through the communication module.

A program capable of controlling the ultrasonic imaging apparatus 40 may be installed in the external device, and this program may include instructions that perform part or all of operations of the processor 50.

The program may be pre-installed in the external device, or a user of the external device may download and install the program from a server providing an application. The server providing the application may include a recording medium in which the program is stored.

The memory 60 may store various data or programs for driving and controlling the ultrasonic imaging apparatus 40, inputted and outputted ultrasonic data, ultrasonic images, etc.

The input interface 170 may receive a user input for controlling the ultrasonic imaging apparatus 40. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

FIG. 1B illustrates a control block diagram of the ultrasonic imaging system in a case in which the probe is a wireless probe or a hybrid probe.

According to various embodiments, the ultrasonic imaging apparatus 40 illustrated in FIG. 1B may be replaced with the ultrasonic imaging apparatus 40 described with reference to FIG. 1A.

According to various embodiments, the probe 20 illustrated in FIG. 1A may be replaced with the probe 20 to be described with reference to FIG. 1B.

The probe 20 may include the transmission module 113, a battery 114, the transducer 117, a charging module 116, the reception module 115, a processor 310, and a communication module 119. Although FIG. 1B illustrates that the probe 20 includes both the transmission module 113 and the reception module 115, the probe 20 may include only part of a configuration of the transmission module 113 and the reception module 115 depending on the implementation type, and the part of the configuration of the transmission module 113 and the reception module 115 may be included in the ultrasonic imaging apparatus 40. Alternatively, the probe 20 may further include the image processor 70.

The transducer 117 may include a plurality of transducers. The plurality of transducers may transmit an ultrasonic signal to the object 10 in response to a transmission signal applied from the transmission module 113. The plurality of transducers may also receive the ultrasonic signal reflected from the object 10 to form a reception signal.

The charging module 116 may charge the battery 114. The charging module 116 may receive electric power from the outside. The charging module 116 may receive electric power wirelessly. However, the disclosure is not limited thereto, and the charging module 116 may also receive electric power through a wire. The charging module 116 may transfer the received electric power to the battery 114.

The processor 310 controls the transmission module 113 to form a transmission signal to be applied to each of the plurality of transducers in consideration of the positions and focused points of the plurality of transducers.

The processor 310 controls the reception module 115 to generate ultrasonic data by converting reception signals received from the transducer 117 into analog to digital and summing up the digitally converted reception signals in consideration of the positions and focused points of the plurality of transducers. Alternatively, in a case in which the probe 20 includes the image processor 70, the probe 20 may generate an ultrasonic image using the generated ultrasonic data.

In the case in which the probe 20 is implemented as a two-dimensional probe, the processor 310 may calculate the time delay value for digital beamforming for each sub-array for each of the plurality of sub-arrays included in the two-dimensional transducer array. The processor 310 may also calculate the time delay value for analog beamforming for each of the transducers included in one of the plurality of sub-arrays. The processor 310 may control the analog beamformer and the digital beamformer to form a transmission signal to be applied to each of the plurality of transducers depending on the time delay values for analog beamforming and the time delay values for digital beamforming. The processor 310 may also control the analog beamformer to sum up the signals received from the plurality of transducers for each sub-array depending on the time delay values for analog beamforming. The processor 310 may also control the ultrasonic transmission/reception module 111 to convert the summed signal for each sub-array into analog to digital. The processor 310 may also control the digital beamformer to generate ultrasonic data by summing up the digitally converted signals depending on the time delay values for digital beamforming.

The processor 310 may control the overall operation of the probe 20 and the signal flows between the internal components of the probe 20. The processor 310 may perform or control various operations or functions of the probe 20 by executing the programs or instructions stored in memory 320. The processor 310 may also control the operation of the probe 20 by receiving a control signal from the input interface 170 of the probe 20 or an external device (e.g., the ultrasonic imaging apparatus 40).

The communication module 119 may wirelessly transmit the generated ultrasonic data or ultrasonic images to the ultrasonic imaging apparatus 40 through a wireless network. The communication module 119 may also receive a control signal and data from the ultrasonic imaging apparatus 40.

The ultrasonic imaging apparatus 40 may receive the ultrasonic data or ultrasonic images from the probe 20.

In an embodiment, in the case in which the probe 20 includes the image processor 70 capable of generating an ultrasonic image using the ultrasonic data, the probe 20 may transmit the ultrasonic data and/or the ultrasonic image generated by the image processor 70 to the ultrasonic imaging apparatus 40.

In an embodiment, in a case in which the probe 20 does not include the image processor 70 capable of generating an ultrasonic image using the ultrasonic data, the probe 20 may transmit the ultrasonic data to the ultrasonic imaging apparatus 40. The ultrasonic data may include ultrasonic raw data, and the ultrasonic image may refer to ultrasonic image data.

The ultrasonic imaging apparatus 40 may include the processor 50, the image processor 70, the display 150, the memory 60, the communication module 160, and the input interface 170.

The image processor 70 generates an ultrasonic image using the ultrasonic data received from the probe 20.

The display 150 may display the ultrasonic image received from the probe 20, an ultrasonic image generated by processing the ultrasonic data received from the probe 20, and a variety of information processed in the ultrasonic imaging system 1. The ultrasonic imaging apparatus 40 may include the one or more displays 150 depending on the implementation type. The display 150 may also include a touch panel or a touch screen.

The processor 50 may control the overall operation of the ultrasonic imaging apparatus 40 and the signal flows between the internal components of the ultrasonic imaging apparatus 40. The processor 50 may perform or control various operations or functions of the ultrasonic imaging apparatus 40 by executing the programs or applications stored in the memory 60. The processor 50 may also control the operation of the ultrasonic imaging apparatus 40 by receiving a control signal from the input interface 170 or an external device.

The ultrasonic imaging apparatus 40 may include the communication module 160, and may be connected with the external device (e.g., the probe 20, a server, a medical device, a portable device (a smart phone, tablet PC, wearable device, etc.)) through the communication module 160.

The communication module 160 may include one or more components enabling communication with the external device, and may include, for example, at least one of a short-range communication module, a wired communication module, and a wireless communication module.

The communication module 160 of the ultrasonic imaging apparatus 40 and the communication module 119 of the probe 20 may communicate using a network or a short-range wireless communication method. For example, the communication module 160 of the ultrasonic imaging apparatus 40 and the communication module 119 of the probe 20 may communicate using any one of wireless LAN, Wi-Fi, Bluetooth, ZigBee, Wi-Fi Direct (WFD), Infrared Data Association (IrDA), Bluetooth Low Energy (BLE), Near Field Communication (NFC), Wireless Broadband Internet (WiBro), World Interoperability for Microwave Access (WiMAX), Shared Wireless Access Protocol (SWAP), Wireless Gigabit Alliance (WiGig), RF communication, and a wireless data communication method including 60 GHz millimeter wave (mm wave) short-range communication.

To this end, the communication module 160 of the ultrasonic imaging apparatus 40 and the communication module 119 of the probe 20 may include at least one of a wireless LAN communication module, a Wi-Fi communication module, a Bluetooth communication module, a ZigBee communication module, a Wi-Fi Direct (WFD) communication module, an Infrared Data Association (IrDA) communication module, a Bluetooth Low Energy (BLE) communication module, a Near Field Communication (NFC) module, a Wireless Broadband Internet (WiBro) communication module, a World Interoperability for Microwave Access (WiMAX) communication module, a Shared Wireless Access Protocol (SWAP) communication module, a Wireless Gigabit Alliance (WiGig) communication module, a RF communication module, and a 60 GHz millimeter wave (mm wave) short-range communication module.

In an embodiment, the probe 20 may transmit device information (e.g., ID information) of the probe 20 to the ultrasonic imaging apparatus 40 using a first communication method (e.g., BLE) and be wirelessly paired with the ultrasonic imaging apparatus 40, and may transmit ultrasonic data and/or ultrasonic images to the paired ultrasonic imaging apparatus 40.

The device information of the probe 20 may include a variety of information related to a serial number, model name, and battery state of the probe 20.

The ultrasonic imaging apparatus 40 may receive the device information (e.g., ID information) of the probe 20 from the probe 20 using the first communication method (e.g., BLE) and be wirelessly paired with the probe 20, and may transmit an activation signal to the paired probe 20 and receive the ultrasonic data and/or ultrasonic images from the probe 20. In this case, the activation signal may include a signal for controlling the operation of the probe 20.

In an embodiment, the probe 20 may transmit the device information (e.g., ID information) of the probe 20 to the ultrasonic imaging apparatus 40 using the first communication method (e.g., BLE) and be wirelessly paired with the ultrasonic imaging apparatus 40, and may transmit the ultrasonic data and/or ultrasonic images to the ultrasonic imaging apparatus 40 paired by the first communication method using a second communication method (e.g., 60 GHz millimeter wave and Wi-Fi).

The ultrasonic imaging apparatus 40 may receive the device information (e.g., ID information) of the probe 20 from the probe 20 using the first communication method (e.g., BLE) and be wirelessly paired with the probe 20, and transmit the activation signal to the paired probe 20 and receive the ultrasonic data and/or ultrasonic images from the probe 20 using the second communication method (e.g., 60 GHz millimeter wave and Wi-Fi).

According to various embodiments, the first communication method used to pair the probe 20 and the ultrasonic imaging apparatus 40 with each other may have a frequency band lower than a frequency band of the second communication method used by the probe 20 to transmit the ultrasonic data and/or ultrasonic images to the ultrasonic imaging apparatus 40.

The display 150 of the ultrasonic imaging apparatus 40 may display user interfaces (UIs) indicating the device information of the probe 20. For example, the display 150 may display Uls, which indicate identification information of the wireless probe 20, a pairing method of indicating a pairing method with the probe 20, a data communication state between the probe 20 and the ultrasonic imaging apparatus 40, a method of performing data communication with the ultrasonic imaging apparatus 40, and the battery state of the probe 20.

In a case in which the probe 20 includes the display 150, the display 150 of the probe 20 may display Uls indicating the device information of the probe 20. For example, the display 150 may display UIs, which indicate the identification information of the wireless probe 20, a pairing method of indicating the pairing method with the probe 20, the data communication state between the probe 20 and the ultrasonic imaging apparatus 40, the method of performing the data communication with the ultrasonic imaging apparatus 40, and the battery state of the probe 20.

The communication module 160 may receive a control signal and data from an external device and transmit the received control signal to the processor 50 so that the processor 50 controls the ultrasonic imaging apparatus 40 in response to the received control signal.

Alternatively, the processor 50 may transmit a control signal to the external device through the communication module 160 to control the external device according to the control signal of the processor 50.

For example, the external device may process the data of the external device according to the control signal of the processor 50 received through the communication module.

A program capable of controlling the ultrasonic imaging apparatus 40 may be installed in the external device, and this program may include instructions that perform part or all of operations of the processor 50.

The program may be pre-installed in the external device, or a user of the external device may download and install the program from a server providing an application. The server providing the application may include a recording medium in which the program is stored.

The memory 60 may store various data or programs for driving and controlling the ultrasonic imaging apparatus 40, inputted and outputted ultrasonic data, ultrasonic images, etc.

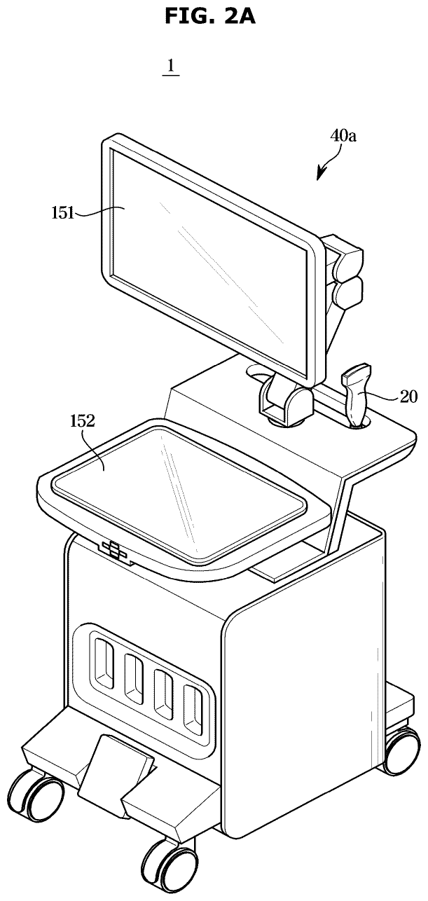

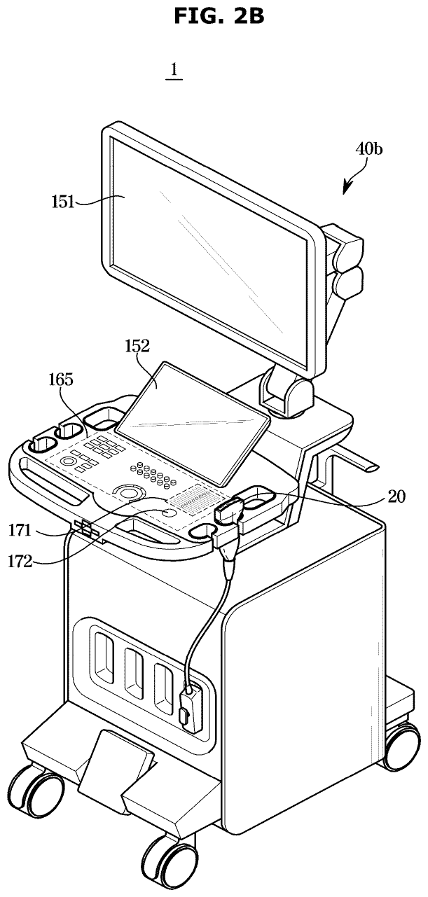

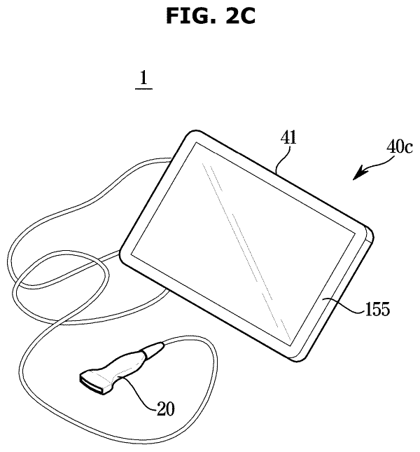

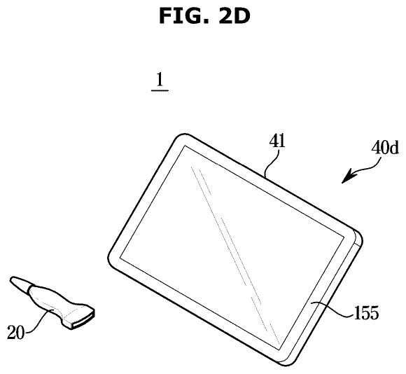

Examples of the ultrasonic imaging system 1 according to an embodiment of the disclosure will be described later through FIGS. 2A, 2B, 2C, and 2D.

FIGS. 2A, 2B, 2C, and 2D are views illustrating ultrasonic imaging apparatuses 40a, 40b, 40c, and 40d according to an embodiment.

Referring to FIGS. 2A and 2B, ultrasonic imaging apparatuses 40a and 40b may include a main display 151 (150) and a sub display 152 (150). At least one of the main display 151 and the sub display 152 may be implemented as a touch screen. At least one of the main display 151 and the sub display 152 may display ultrasonic images or a variety of information processed in the ultrasonic imaging apparatuses 40a and 40b. In addition, at least one of the main display 151 and the sub display 152 may be implemented as a touch screen and provide graphic user interfaces (GUIs), so that data for controlling the ultrasonic imaging apparatuses 40a and 40b may be inputted thereinto from a user. For example, the main display 151 may display ultrasonic images, and the sub display 152 may display a control panel for controlling the display of the ultrasonic images in the form of GUIs. Data for controlling the display of images may be inputted into the sub display 152 through the control panel displayed in the form of GUIs. For example, a time gain compensation (TGC) button, a Freeze button, a trackball, a jog switch, a knob, and the like may be provided as GUI on the sub display 152.

The ultrasonic imaging apparatuses 40a and 40b may control the display of ultrasonic images displayed on the main display 151 using the inputted control data. The ultrasonic imaging apparatuses 40a and 40b may also be connected to the probe 20 wire or wirelessly to transmit and receive ultrasonic signals to and from the object 10.

Referring to FIG. 2B, the ultrasonic imaging apparatus 40b may further include a control panel 165 in addition to the main display 151 and the sub display 152. The control panel 165 may include a button, a trackball, a jog switch, a knob, and the like, and data for controlling the ultrasonic imaging apparatus 40b may be inputted into the control panel 165 from the user. For example, the control panel 165 may include a TGC button 171, a Freeze button 172, and the like. The TGC button 171 is a button for setting a TGC value for each depth of the ultrasonic images. When the input of the Freeze button 172 is sensed while scanning an ultrasonic image, the ultrasonic imaging apparatus 40b may maintain a state in which a frame image at that point in time is displayed.

The button, the trackball, the jog switch, the knob, and the like included in the control panel 165 may be provided as GUIs on the main display 151 or the sub display 152. The ultrasonic imaging apparatuses 40a and 40b may be connected to the probe 20 to transmit and receive ultrasonic signals to and from the object 10.

Referring to FIGS. 2C and 2D, ultrasonic imaging apparatuses 40c and 40d may be implemented in a portable type. The portable ultrasonic imaging apparatuses 40c and 40d may include, for example, smart phones, laptop computers, PDAs, tablet PCs, and the like, which include probes and applications, but is not limited thereto.

The ultrasonic imaging apparatus 40c may include a main body 41. Referring to FIG. 2C, the probe 20 may be connected to one side of the main body 41 by wire. To this end, the main body 41 may include a connection terminal to and from which a cable connected to the probe 20 may be attached and detached, and the probe 20 may include a connection terminal to and from which a cable connected to the main body 41 may be attached and detached.

Referring to FIG. 2D, the probe 20 may be wirelessly connected to the ultrasonic imaging apparatus 40d. The main body 41 may include an input/output interface (e.g., a touch screen) 155 (150 and 170). Ultrasonic images, a variety of information processed by the ultrasonic imaging apparatus, GUIs, and the like may be displayed on the input/output interface 155.

In addition, an ultrasonic image may be displayed on the input/output interface 155. The ultrasonic imaging apparatus 40d may correct the ultrasonic image displayed on the input/output interface 155 using AI. The ultrasonic imaging apparatus 40d may provide an alarm that notifies information about a lesion in the ultrasonic image displayed on the input/output interface 155 using various audiovisual tools such as graphics, sound, and vibration, using AI.

The ultrasonic imaging apparatus 40d may output a control panel displayed in GUI form through the input/output interface 155.

The ultrasonic imaging system 1 according to the disclosure may include the probe 20 configured to emit an ultrasonic signal and the ultrasonic imaging apparatus 40 configured to wirelessly communicate with the probe 20.

The one transducer 117 emitting an ultrasonic signal is provided on each of both sides of the probe 20, so that the probe 20 may include a total of the two transducers 117. This structure of the probe 20 is referred to as a dual head structure. The probe 20 may include transmitter (not shown) including the transmission module 113 and a receiver 600 including the reception module 115. The transmitter (not shown) may generate a transmission signal that is transmitted to the probe 20 to obtain a frame of an ultrasonic image. The frame of an ultrasonic image may include a frame such as an amplitude mode (A-mode), a brightness mode (B-mode), a color mode (C-mode), a Doppler mode (D-mode), an elastography mode (E-mode), a motion mode (M-mode), and an elasticity image.

The probe 20 may convert the transmission signal into an ultrasonic signal and emit the converted ultrasonic signal to a target region inside object 10. The probe 20 may receive an echo signal, which is a reflection signal of the ultrasonic signal, and convert the received echo signal to generate a reception signal. The receiver 600 may receive the converted reception signal from the probe 20.

To this end, the probe 20 may include the transducer 117 and a MUX circuit. The transducer 117 may vibrate to convert an electrical signal into an ultrasonic signal or an ultrasonic signal to an electrical signal.

The transducer 117 of the probe 20 may be implemented as a piezoelectric ultrasonic transducer using the piezoelectric effect. To this end, the transducer 117 may include a piezoelectric material or a piezoelectric thin film. When an alternating current is applied to a piezoelectric material or a piezoelectric thin film from an internal electric storage device such as a battery or an external power supply, the piezoelectric material or the piezoelectric thin film vibrates at a predetermined frequency, and an ultrasonic signal of a predetermined frequency is generated according to the vibration frequency.

When an echo signal of a predetermined frequency reaches the piezoelectric material or the piezoelectric thin film, the piezoelectric material or the piezoelectric thin film vibrates according to the frequency of the arrived echo signal, and the piezoelectric material or the piezoelectric thin film outputs an alternating current of a frequency corresponding to the vibration frequency.

The transducer 117 of the probe 20 may also be implemented by other transducers, such as a magnetostrictive ultrasonic transducer, which utilizes the magnetostrictive effect of a magnetic material, or a capacitive micromachined ultrasonic transducer (cMUT), which transmits and receives ultrasonic waves using the vibration of hundreds or thousands of micromachined thin films.

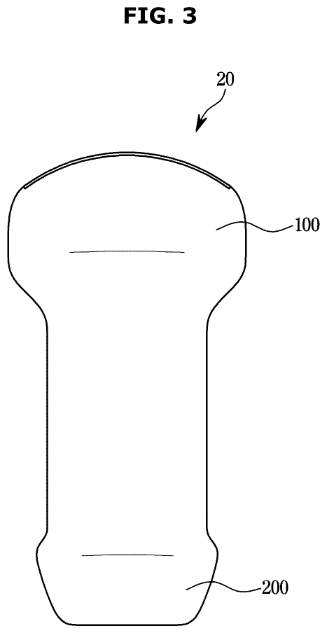

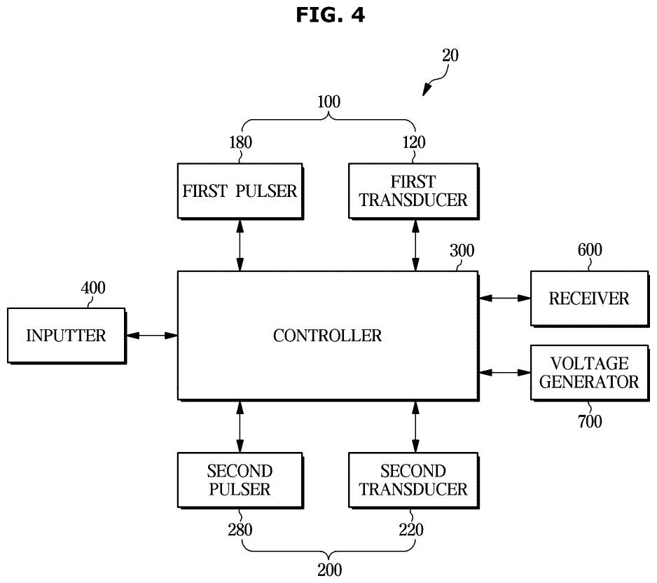

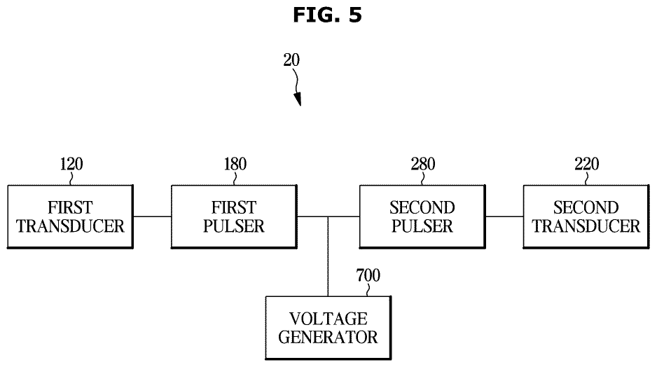

FIG. 3 is a view illustrating an external appearance of an ultrasonic probe according to an embodiment, FIG. 4 is a control block diagram of the ultrasonic probe according to an embodiment, and FIG. 5 is a diagram schematically illustrating a circuit structure of the ultrasonic probe according to an embodiment.

Referring to FIG. 3, the probe 20 may be configured as a dual head structure. The probe 20 may include a first head 100 provided on a first side and a second head 200 provided on a second side.

Referring to FIGS. 3, 4 and 5, the probe 20 may include a first transducer 120, a first pulser 180, a second transducer 220, a second pulser 280, an inputter 400, the receiver 600, a voltage generator 700, and a controller 300 configured to control the aforementioned configuration.

The probe 20 of the ultrasonic imaging system 1 according to the disclosure may be configured as a dual head structure. Each of the heads 100 and 200 may include a transducer.

The first head 100 of the probe 20 may include the first transducer 120 and the first pulser 180. The second head 200 of the probe 20 may include the second transducer 220 and the second pulser 280. The receiver 600 may be provided between the first head 100 and the second head 200. However, the receiver 600 may be provided with different arrangements and structures depending on an intention of a designer.

The first transducer 120 may include at least one first transducer element, and the first transducer element may convert an electrical signal and an ultrasonic signal into each other. The first transducer element may include the first transducer 120 array consisting of at least one row and at least one column.

The first transducer 120 may convert a first transmission signal transmitted from a transmitter (not shown) into a first ultrasonic signal. The first transducer 120 may emit the converted first ultrasonic signal to the object 10. Specifically, the first transducer 120 may emit the first ultrasonic signal to the target region inside the object 10. The first transmission signal may be an electrical signal.

The first transducer 120 may receive a first echo signal in which the emitted first ultrasonic signal is reflected from the target region inside the object 10. The first transducer 120 may generate and output a first reception signal based on the received first echo signal. The generated first reception signal may be transmitted to the receiver 600.

The at least one first transducer element included in the first transducer 120 may be arranged on one surface of a first housing of the first transducer 120. Specifically, the at least one first transducer element may be arranged in a direction parallel to a first opening so that transmission and reception of the first ultrasonic signal and the first echo signal may be performed through the first opening provided on the one surface of the first housing.

The receiver 600 may receive the first reception signal output from the first transducer 120. The first reception signal may correspond to a low voltage signal compared to the first transmission signal, which is a high voltage signal. Therefore, generally, the receiver 600 may use a range corresponding to a voltage of the first reception signal generated from the first transducer 120 as an input range.

Each of one or more shared reception elements may receive the first reception signal from each of the one or more first transducer elements.

The first pulser 180 may transmit an electrical signal to the first transducer 120 so that an ultrasonic signal may be generated in the first transducer 120.

The second transducer 220 may include at least one second transducer element, and the second transducer element may convert an electrical signal and an ultrasonic signal into each other. The second transducer element may include the second transducer 220 array consisting of at least one row and at least one column.

The second transducer 220 may convert a second transmission signal transmitted from a transmitter (not shown) into a second ultrasonic signal. The second transducer 220 may emit the converted second ultrasonic signal to the object 10. Specifically, the second transducer 220 may emit the second ultrasonic signal to the target region inside the object 10. The second transmission signal may be an electrical signal.

The second transducer 220 may receive a second echo signal in which the emitted second ultrasonic signal is reflected from the target region inside the object 10. The second transducer 220 may generate and output a second reception signal based on the received second echo signal. The generated second reception signal may be transmitted to the receiver 600.

The at least one second transducer element included in the second transducer 220 may be arranged on one surface of a second housing of the second transducer 220. Specifically, the at least one second transducer element may be arranged in a direction parallel to a second opening so that transmission and reception of the second ultrasonic signal and the second echo signal may be performed through the second opening provided on the one surface of the second housing.

The receiver 600 may receive the second reception signal output from the second transducer 220. The second reception signal may correspond to a low voltage signal compared to the second transmission signal, which is a high voltage signal. Therefore, generally, the receiver 600 may use a range corresponding to a voltage of the second reception signal generated from the second transducer 220 as an input range.

Each of one or more shared reception elements may receive the second reception signal from each of the one or more second transducer elements.

The second pulser 280 may transmit an electrical signal to the second transducer 220 so that an ultrasonic signal may be generated in the second transducer 220.

The voltage generator 700 may generate a voltage and supply the voltage to the first pulser 180 and the second pulser 280 so that the first pulser 180 and the second pulser 280 may supply electrical signals. The voltage generator 700 may include a storage module, such as a capacitor, for storing electrical energy generated to supply to the first pulser 180 and the second pulser 280.

When an image mode is changed in a state in which the first transducer 120 is activated, the controller 300 may supply the voltage generated in the voltage generator 700 to the second pulser 280 to discharge the voltage supplied to the first pulser 180. A detailed description of this will be provided later.

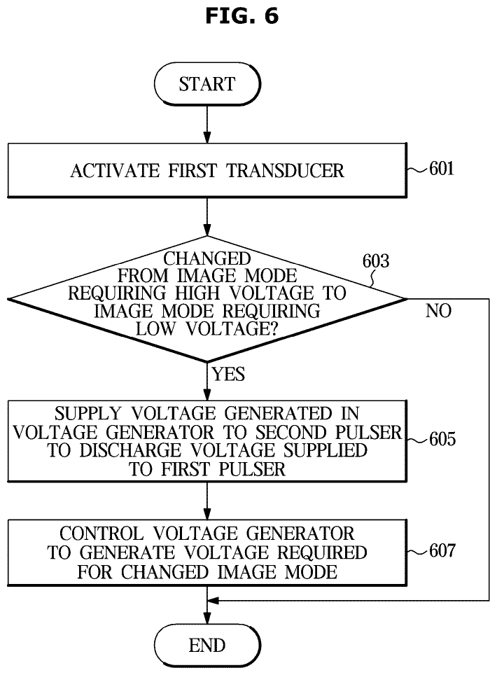

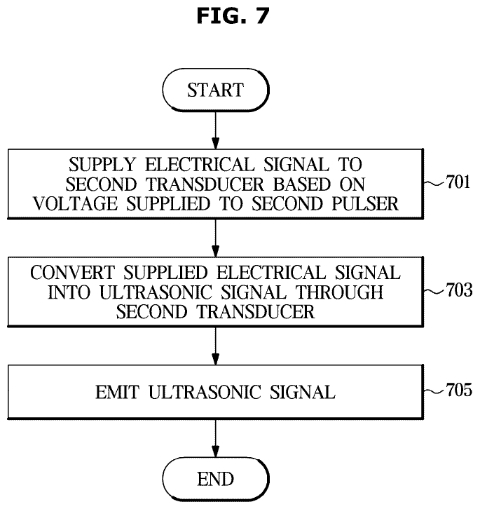

FIGS. 6 and 7 are flowcharts illustrating control methods of the ultrasonic probe according to an embodiment.

As described above, an ultrasonic image may include images of various modes such as the amplitude mode (A-mode), the brightness mode (B-mode), the color mode (C-mode), the Doppler mode (D-mode), the elastography mode (E-mode), the motion mode (M-mode), and the elasticity image.

In a case in which an ultrasonic image in a specific mode is changed to an ultrasonic image in another mode, the required voltages may be different such that an appropriate image may be displayed in each image mode.

In a case of being changed from an image mode requiring a relatively higher voltage as an appropriate voltage to an image mode requiring a relatively lower voltage as an appropriate voltage among the required voltages, a voltage generated by the voltage generator to be supplied to the pulser needs to be lowered. In this case, when the voltage stored in the storage module, such as a capacitor, included in the voltage generator 700, is discharged using a separate discharge module, a volume and weight increase due to the separate discharge module, and heat generation may occur due to the discharge module.

In order to solve this problem, in the disclosure, a voltage generated in the voltage generator 700 and stored in the storage module such as a capacitor in order to supply the voltage to a transducer and a pulser, which are being activated, may be transmitted to a pulser and a transducer, which are being deactivated, and the voltage may be converted into ultrasonic energy and released to be rapidly discharged without a separate discharge module.

Hereinafter, it will be explained assuming that a transducer which is being activated is the first transducer 120.

That is, in the case of being changed from an image mode requiring a relatively higher voltage as an appropriate voltage to an image mode requiring a relatively lower voltage as an appropriate voltage (YES in 603) in the state in which the first transducer 120 is activated (601), a voltage generated in the voltage generator 700 in order to supply the voltage to the first pulser 180 may be supplied to the second pulser 280 to discharge a voltage supplied to the first pulser 180 (605).

Specifically, in a case in which a voltage to be supplied to the first pulser 180 needs to be lowered due to a change of an image mode (e.g., 30 [V]) in a situation where the voltage generator 700 supplies a constant voltage (e.g., 60[V]) to the first pulser 180 for a specific image mode, the voltage generated in the voltage generator 700 in order to be supplied to the first pulser 180 may be supplied to the second pulser 280, which is being inactivated, so that the second pulser 280 may be driven.

The controller 300 may set the voltage generated in the voltage generator 700 to a voltage (e.g., 30[V]) suitable for the changed image mode before supplying the voltage to the second pulser 280.

Accordingly, the voltage generator 700 may supply the voltage to the second pulser 280 and drive the second pulser 280 to generate a voltage (e.g., 30[V]) suitable for the changed image mode after releasing all the existing voltage stored in the storage module such as a capacitor within the voltage generator 700 (607).

According to this control, when changed to an image mode requiring a lower voltage than the existing voltage as an appropriate voltage, the charged electric energy of the voltage generator may be discharged without a separate discharge module, so that the image mode may be rapidly changed.

The controller 300 may control the second transducer 220 to emit an ultrasonic signal based on the voltage supplied to the second pulser 280.

That is, an electrical signal may be supplied to the second transducer 220 based on the voltage supplied to the second pulser 280 (701), and the second transducer 220 may convert the supplied electrical signal into an ultrasonic signal (703) and emit the ultrasonic signal (705).

As such, as electrical energy supplied to the first head 100, which is being activated, is supplied to the second head 200, which is being deactivated, and is released as ultrasonic energy, the voltage supplied to the first pulser 180 may be discharged more rapidly without a separate module.

Additionally, because the second head 200, which is being deactivated, faces in the opposite direction of the object 10, the ultrasonic energy emitted from the second head 200 may be emitted to the outside rather than toward the object 10.

Although not shown in the drawings, the ultrasonic probe 20 may further include a third head (not shown) as another embodiment, and the third head may include a third transducer (not shown) and a third pulser (not shown).

In this case, in a case of being changed from an image mode requiring a relatively higher voltage as an appropriate voltage to an image mode requiring a relatively lower voltage as an appropriate voltage in the state in which the first transducer 120 is activated, the controller 300 may supply the voltage generated in the voltage generator 700 to the second pulser 280 and the third pulser to discharge the voltage supplied to the first pulser 180.

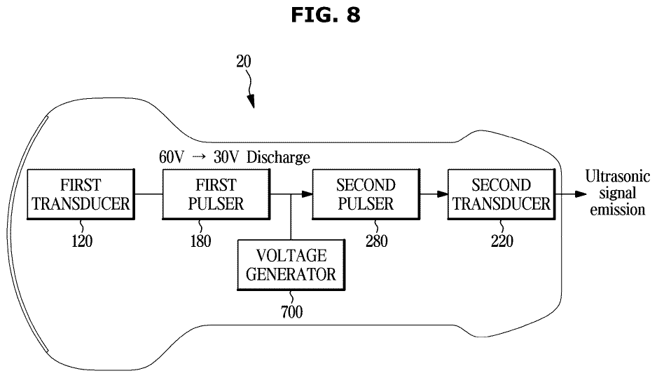

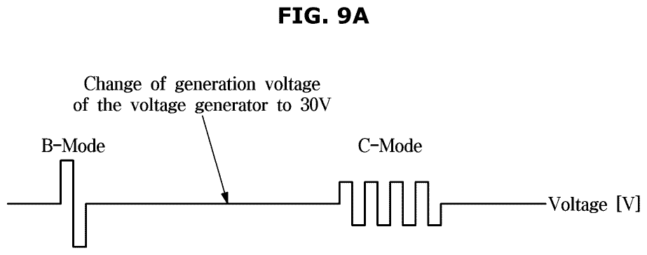

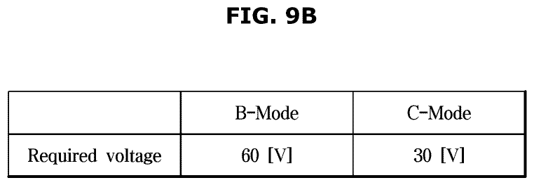

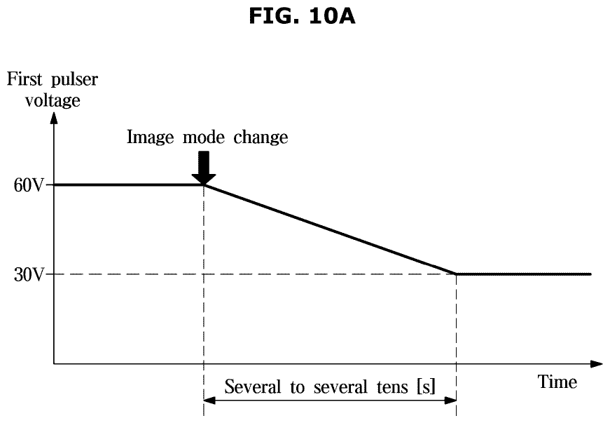

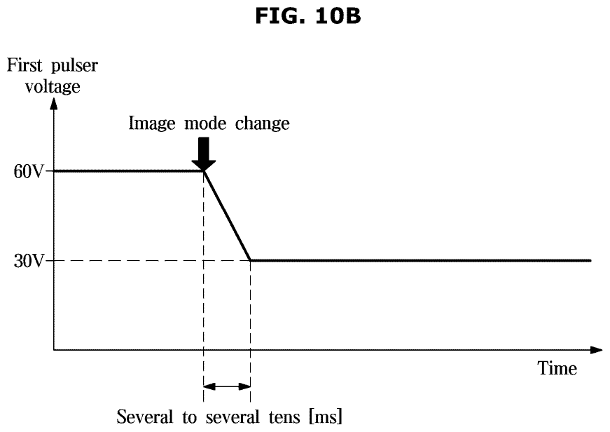

FIG. 8 is a diagram for explaining a movement of an electrical signal according to a change of an image mode according to an embodiment, FIGS. 9A and 9B are diagrams for explaining a required voltage according to the change of the image mode according to an embodiment, and FIGS. 10A and 10B are graphs for explaining a discharge time of a pulsar according to an embodiment.

As described above, the disclosure is intended to facilitate rapid discharge in the case of being changed from an image mode requiring a relatively higher voltage (first voltage) as an appropriate voltage to an image mode requiring a relatively lower voltage (second voltage) as an appropriate voltage,

Additionally, the disclosure may be applied whenever an image mode requiring a first voltage and an image mode requiring a second voltage are repeatedly changed, or whenever the voltage is changed from the first voltage to the second voltage when both the image modes are displayed simultaneously.

Herein, an image mode requiring a relatively higher voltage (first voltage) may be, for example, the B-mode. In the case of the B-mode, the required appropriate voltage may be 60 to 70 [V]. Also, an image mode requiring a relatively lower voltage (second voltage) may be, for example, the C-mode or D-mode. In the case of the C-mode, the required appropriate voltage may be 30 [V], and in the case of the D-mode, the required appropriate voltage may be 5 to 30 [V].

That is, because as illustrated in FIGS. 9A and 9B, when the image mode is changed from the B-mode image to the C-mode image, the required appropriate voltage is lowered from 60 [V] to 30 [V], the voltage stored and supplied in and to the storage module of the voltage generator 700, such as a capacitor, needs to be lowered to 30 [V].

Accordingly, as illustrated in FIG. 8, electrical energy stored in the storage module of the voltage generator 700, such as a capacitor, may be transferred to the second pulser 280 in order to be supplied to the first pulser 180, the electrical energy may be supplied to the second transducer 220, and the electrical energy may be converted into ultrasonic energy and emitted in and from the second transducer 220. Compared to the time it may take several to several tens [s] when the voltage stored in the storage module of the voltage generator 700, such as a capacitor, is naturally discharged without a separate discharge module and a discharge operation, as illustrated in FIG. 10A, a very short time of several to several tens [ms] may be required according to a discharge operation of the disclosure, as illustrated in FIG. 10B compared to the natural discharge time.

An ultrasonic probe according to an embodiment of the disclosure may include a first transducer configured to transmit a first transmission signal and receive a reflection signal, a second transducer configured to transmit a second transmission signal and receive a reflection signal, a first pulser configured to supply an electrical signal to the first transducer, a second pulser configured to supply an electrical signal to the second transducer, a voltage generator configured to supply a voltage to the first pulser and the second pulser, and a controller configured to supply a voltage generated in the voltage generator to the second pulser to discharge a voltage supplied to the first pulser when an image mode is changed in a state in which the first transducer is activated.

According to the disclosure, the voltage generator can be discharged without a separate discharge module so that the image mode can be rapidly changed.

In addition, because there is no discharge module, a volume and weight occupied by the ultrasonic probe can be reduced, and a voltage suitable for the changed image mode can be applied, so that the image quality can be secured.

The controller may be configured to control the second transducer to emit an ultrasonic signal based on the voltage supplied to the second pulser.

The controller may be configured to supply the electrical signal to the second transducer based on the voltage supplied to the second pulser, and control the second transducer to convert the supplied electrical signal into an ultrasonic signal and emit the converted ultrasonic signal.

The controller may be configured to control the voltage generator to generate a voltage required for the changed image mode after discharging the voltage supplied to the first pulser.

The change of the image mode may include changing from a first image mode requiring a first voltage to a second image mode requiring a second voltage lower than the first voltage.

The change of the image mode may include repeatedly changing the first image mode and the second image mode.

The first image mode may include a brightness mode (B-mode), and the second image mode may include a color mode (C-mode) or a Doppler mode (D-mode). The ultrasonic probe may further include an inputter configured to receive a user input, wherein the controller may change the image mode based on the user input received by the inputter.

The ultrasonic probe may further include a third transducer configured to transmit a third transmission signal and receive a reflection signal and a third pulser configured to supply the electrical signal to the third transducer, wherein the controller may be configured to supply the voltage generated in the voltage generator to the second pulser and the third pulser to discharge the voltage supplied to the first pulser when the image mode is changed in the state in which the first transducer is activated.

A control method of an ultrasonic probe according to an embodiment of the disclosure, which includes a first transducer configured to transmit a first transmission signal and receive a reflection signal, a second transducer configured to transmit a second transmission signal and receive a reflection signal, a first pulser configured to supply an electrical signal to the first transducer, a second pulser configured to supply an electrical signal to the second transducer, and a voltage generator configured to supply a voltage to the first pulser and the second pulser, may include activating the first transducer, receiving a command for a change of an image mode, and supplying a voltage generated in the voltage generator to the second pulser to discharge a voltage supplied to the first pulser.

The control method may further include controlling the second transducer to emit an ultrasonic signal based on the voltage supplied to the second pulser.

The controlling of the second transducer to emit the ultrasonic signal may include supplying the electrical signal to the second transducer based on the voltage supplied to the second pulser, and controlling the second transducer to convert the supplied electrical signal into an ultrasonic signal and emit the converted ultrasonic signal.

The control method may further include controlling the voltage generator to generate a voltage required for the changed image mode after discharging the voltage supplied to the first pulser.

The change of the image mode may include changing from a first image mode requiring a first voltage to a second image mode requiring a second voltage lower than the first voltage.

The change of the image mode may include repeatedly changing the first image mode and the second image mode.

The first image mode may include a brightness mode (B-mode), and the second image mode may include a color mode (C-mode) or a Doppler mode (D-mode). The ultrasonic probe may further include an inputter configured to receive a user input, and the receiving of the command for the change of the image mode may include receiving a command for a change of the image mode based on the user input received by the inputter.

According to the disclosure, the pulser can be discharged without a separate discharge module so that the image mode can be rapidly changed.

In addition, because there is no discharge module, a volume and weight occupied by the ultrasonic probe can be reduced, and a voltage suitable for the changed image mode can be applied, so that the image quality can be secured.

The disclosed embodiments may be implemented in the form of a recording medium storing instructions executable by a computer. The instructions may be stored in the form of program code, and when executed by a processor, a program module may be created to perform the operations of the disclosed embodiments.

The device-readable recording medium may be provided in the form of a non-transitory storage medium. Herein, the ‘non-transitory storage medium’ simply means that it is a tangible device and does not contain signals (e.g. electromagnetic waves), and this term does not distinguish between a case in which data is semi-permanently stored in a storage medium and a case in which data is stored temporarily. For example, the ‘non-transitory storage medium’ may include a buffer in which data is temporarily stored.

According to an embodiment, the methods according to various embodiments disclosed in this document may be included and provided in a computer program product. The computer program product is a commodity and may be traded between sellers and buyers. The computer program product may be distributed in the form of a machine-readable recording medium (e.g., compact disc read only memory (CD-ROM)), or may be distributed (e.g., downloaded or uploaded) online, through an application store (e.g., Play Store™) or directly between two user devices (e.g., smartphones). In the case of online distribution, at least a portion of the computer program product (e.g., a downloadable app) may be at least temporarily stored or created temporarily in the machine-readable recording medium, such as the memory of a manufacturer server, an application store server, and a relay server.

Effects to be achieved in this document are not limited to the effects mentioned above, and other effects not mentioned will be clearly understood by those skilled in the art from the description above.

The foregoing has illustrated and described specific embodiments. However, it should be understood by those of skilled in the art that the disclosure is not limited to the above-described embodiments, and various changes and modifications may be made without departing from the technical idea of the disclosure described in the following claims.

What is claimed is:

1. An ultrasonic probe comprising:
a first transducer configured to transmit a first transmission signal and receive a reflection signal;
a second transducer configured to transmit a second transmission signal and receive a reflection signal;
a first pulser configured to supply an electrical signal to the first transducer;
a second pulser configured to supply an electrical signal to the second transducer;
a voltage generator configured to supply a voltage to the first pulser and the second pulser; and
a controller configured to supply a voltage generated in the voltage generator to the second pulser to discharge a voltage supplied to the first pulser when an image mode is changed in a state in which the first transducer is activated.

2. The ultrasonic probe according to claim 1, wherein
the controller controls the second transducer to emit an ultrasonic signal based on the voltage supplied to the second pulser.

3. The ultrasonic probe according to claim 2, wherein
the controller is configured to supply the electrical signal to the second transducer based on the voltage supplied to the second pulser, and control the second transducer to convert the supplied electrical signal into an ultrasonic signal and emit the converted ultrasonic signal.

4. The ultrasonic probe according to claim 1, wherein
the controller is configured to control the voltage generator to generate a voltage required for the changed image mode after discharging the voltage supplied to the first pulser.

5. The ultrasonic probe according to claim 1, wherein
the change of the image mode comprises changing from a first image mode requiring a first voltage to a second image mode requiring a second voltage lower than the first voltage.

6. The ultrasonic probe according to claim 5, wherein
the change of the image mode comprises repeatedly changing the first image mode and the second image mode.

7. The ultrasonic probe according to claim 6, wherein
the first image mode comprises a brightness mode (B-mode), and the second image mode comprises a color mode (C-mode) or a Doppler mode (D-mode).

8. The ultrasonic probe according to claim 1, further comprising
an inputter configured to receive a user input,
wherein the controller changes the image mode based on the user input received by the inputter.

9. The ultrasonic probe according to claim 1, further comprising:
a third transducer configured to transmit a third transmission signal and receive a reflection signal; and a third pulser configured to supply the electrical signal to the third transducer,
wherein the controller is configured to supply the voltage generated in the voltage generator to the second pulser and the third pulser to discharge the voltage supplied to the first pulser when the image mode is changed in the state in which the first transducer is activated.

10. A control method of an ultrasonic probe, which comprises a first transducer configured to transmit a first transmission signal and receive a reflection signal, a second transducer configured to transmit a second transmission signal and receive a reflection signal, a first pulser configured to supply an electrical signal to the first transducer, a second pulser configured to supply an electrical signal to the second transducer, and a voltage generator configured to supply a voltage to the first pulser and the second pulser, comprising:
activating the first transducer;
receiving a command for a change of an image mode; and
supplying a voltage generated in the voltage generator to the second pulser to discharge a voltage supplied to the first pulser.

11. The control method according to claim 10, further comprising
controlling the second transducer to emit an ultrasonic signal based on the voltage supplied to the second pulser.

12. The control method according to claim 11, wherein
the controlling of the second transducer to emit the ultrasonic signal comprises:
supplying the electrical signal to the second transducer based on the voltage supplied to the second pulser; and
controlling the second transducer to convert the supplied electrical signal into an ultrasonic signal and emit the converted ultrasonic signal.

13. The control method according to claim 10, further comprising
controlling the voltage generator to generate a voltage required for the changed image mode after discharging the voltage supplied to the first pulser.

14. The control method according to claim 10, wherein
the change of the image mode comprises changing from a first image mode requiring a first voltage to a second image mode requiring a second voltage lower than the first voltage.

15. The control method according to claim 14, wherein
the change of the image mode comprises repeatedly changing the first image mode and the second image mode.

16. The control method according to claim 15, wherein
the first image mode comprises a brightness mode (B-mode), and the second image mode comprises a color mode (C-mode) or a Doppler mode (D-mode).

17. The control method according to claim 10, wherein
the ultrasonic probe further comprises an inputter configured to receive a user input, and
the receiving of the command for the change of the image mode comprises receiving a command for a change of the image mode based on the user input received by the inputter.
